(19) United States
(12) Patent Application Publication
(10) Pub. No.: US 2017/0189569 A1
(43) Pub. Date: Jul. 6, 2017

(54) BIODEGRADABLE MICROSPHERES INCORPORATING RADIONUCLIDES TECHNICAL FIELD

(71) Applicants: Omid Souresrafil, Maple Grove, MN (US); Ehson Souresrafil, Maple Grove, MN (US); Nazmi Peyman, Glen Allen, VA (US)

(72) Inventors: Omid Souresrafil, Maple Grove, MN (US); Ehson Souresrafil, Maple Grove, MN (US); Nazmi Peyman, Glen Allen, VA (US)

(21) Appl. No.: 15/460,708

(22) Filed: Mar. 16, 2017

(51) Int. Cl. A61K 51/12; B01J 13/08; A61K 51/06

(52) U.S. Cl. CPC A61K 51/1251; A61K 51/06; A61K 51/1217; B01J 13/08

(57) ABSTRACT

A crosslinked NATURAL POLYMER POLYMER/NATURAL POLYMER POLYMER microsphere comprising a stably incorporated radionuclide. The microsphere can be prepared by droplet microfluidics and used in a method for radiation treatment comprising the administration of microspheres with incorporated radionuclide.

### TECHNICAL FIELD

This invention relates to materials such as microspheres, microdroplets and microparticles, and in turn, to materials that can be used to deliver radionuclides to the body. In another aspect, the invention relates to embolic microspheres formed of multiple crosslinked resorbable polymers.

### BACKGROUND

Many attempts have been made to locally administer radioactive materials to cancer patients as a form of therapy. In some of these, the radioactive materials have been incorporated into small particles, seeds, wires and similar related configurations that can be directly implanted into the cancer. See, for instance, “Treatment of unresectable intrahepatic cholangiocarcinoma with yttrium-90 radioembolization: A systematic review and pooled analysis”, Al-Adra, et al. EJSO J. Cancer Surg. 41(2015):120-127.

Microparticles for such use have taken a variety of forms, and have been made from a similar variety of materials. For example, microspheres are available under the tradenames TheraSphere® Yttrium-90 Glass Microspheres (available from Biocompatibles UK, Ltd, a BTG International company), as well as SIR-Spheres® microspheres, available from Sirtex Medical.

See also PCT application no. WO2002034300A1 (Sirtex Medical) which describes microspheres that are said to comprise a polymer and a stably incorporated radionuclide such as radioactive yttrium, and having a diameter in the range of from 5 to 200 microns. The patent describes a method of preparing such microspheres by step of combining a polymeric matrix and a radionuclide for a time and under conditions sufficient to stably incorporate the radionuclide in the matrix to produce a particulate material.

On a separate subject, processes often referred to as droplet microfluidics have been described that allow the formation of microdroplets from various materials, and for various purposes. One of the key advantages of droplet-based microfluidics is the ability to use droplets as incubators for single cells. See, for instance, Joensson, et al., Droplet Microfluidics—A Tool for Single-Cell Analysis, Angewandte Chemie 51(49):12176-12192, Dec. 3, 2012.

Various techniques have been described for forming polymer microparticles by droplet microfluidics as well. See, for instance, Serra et al., Engineering Polymer Microparticles by Droplet Microfluidics, J. Flow Chem 3(3):66-75 (2013).

On yet another subject, U.S. Pat. No. 8,617,132 (Golzarian, et al) described, inter alia, the preparation and use of embolic materials that generally comprise carboxymethyl chitosan (NATURAL POLYMER POLYMER) crosslinked with Natural Polymer Polymer (NATURAL POLYMER POLYMER). The resulting microspheres can optionally include a therapeutic agent such as doxorubicin.

### SUMMARY

In one aspect, the present invention provides a crosslinked NATURAL POLYMER POLYMER/NATURAL POLYMER POLYMER microsphere comprising a stably incorporated radionuclide. In one preferred aspect, the invention provides a microsphere and incorporated radionuclide prepared by means of droplet microfluidics. In yet another preferred aspect, the invention provides a method for radiation treatment comprising the administration of microspheres with incorporated radionuclide.

The present invention provides microspheres comprising crosslinked NATURAL POLYMER POLYMER/NATURAL POLYMER POLYMER and a radionuclide such as radioactive yttrium. In a preferred embodiment, the microspheres are prepared by the use of droplet microfluidics, and to the use of these microspheres in the treatment of cancer in humans and other mammals.

### BRIEF DESCRIPTION OF THE DRAWINGS

FIG. 1: is a schematic diagram of the preparation technique of the invention.

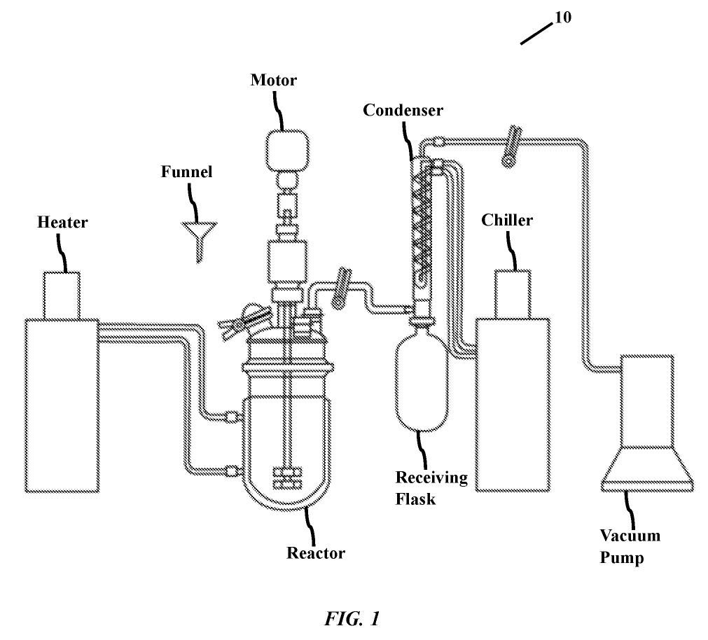

FIG. 2: is a flow chart/schematic view of the process of the invention.

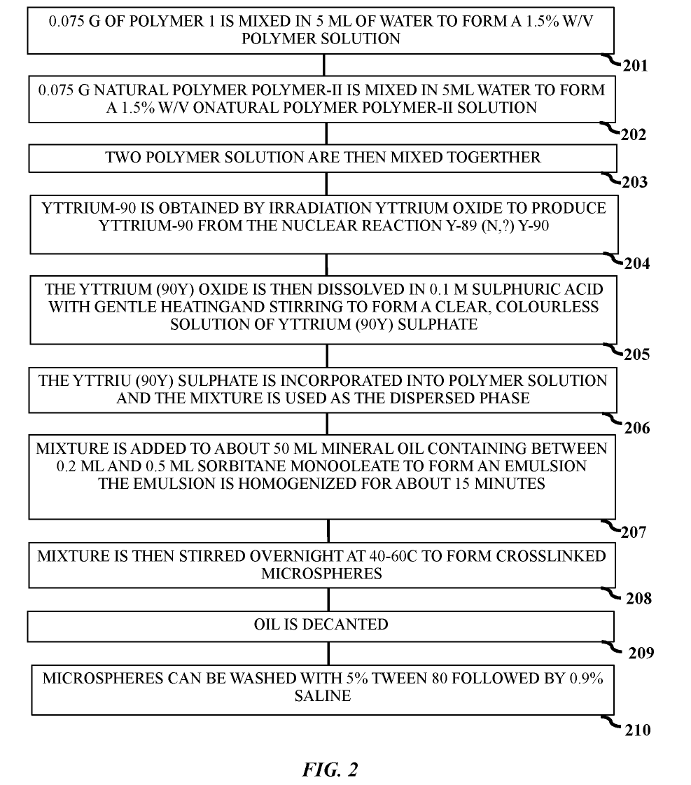

FIG. 3: is a detail schematic view showing the use of droplet microfluidics in microsphere preparation.

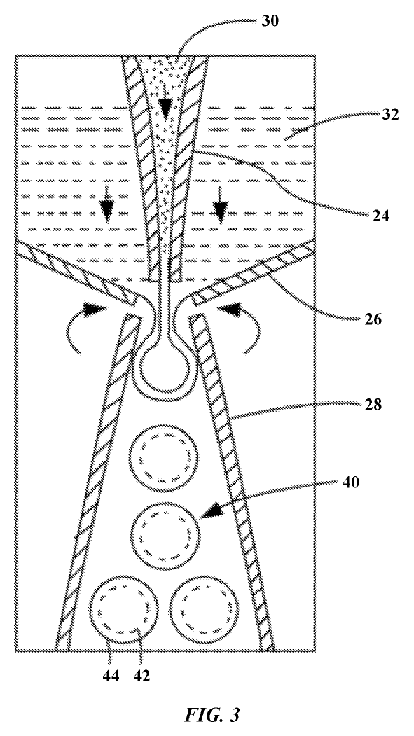

FIG. 4: is a perspective view of a microsphere with parts broken away.

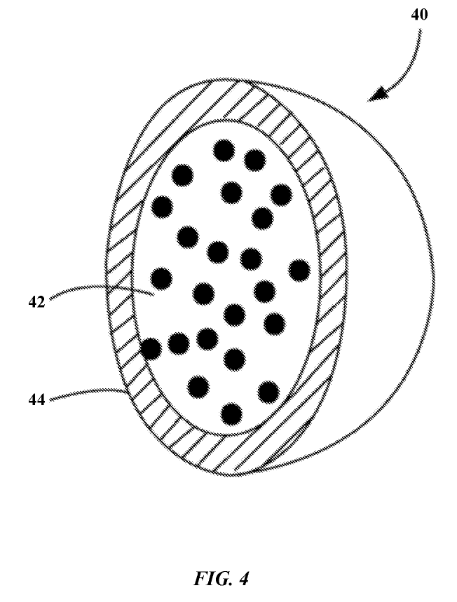

### DETAILED DESCRIPTION

The present disclosure describes a plurality of microspheres that include carboxymethyl chitosan (NATURAL POLYMER POLYMER) crosslinked with Natural Polymer Polymer (NATURAL POLYMER POLYMER). The microspheres are biocompatible, bioresorbable, and biodegradable. In accordance with examples of this disclosure, NATURAL POLYMER POLYMER and NATURAL POLYMER POLYMER may be crosslinked without use of a small molecule crosslinking agent to form microspheres that are substantially free of small molecule crosslinking agent. While the use of a small molecule crosslinking agent facilitates crosslinking reactions, some small-molecule crosslinking agents may be toxic or have other adverse effects on cells or tissue in the body of the patient. By omitting small molecule crosslinking agents, such potential adverse effects may be avoided. In fact, in some examples, the crosslinking reaction between NATURAL POLYMER POLYMER and NATURAL POLYMER POLYMER may be carried out without a small molecule crosslinking agent and at relatively low temperatures (e.g., about 40° C.) in a water and oil emulsion.

NATURAL POLYMER POLYMER is substantially non-toxic and biodegradable. Chitosan breaks down in the body to glucosamine, which can be substantially absorbed by a patient's body. Similarly, NATURAL POLYMER POLYMER is substantially non-toxic and biodegradable. Thus a crosslinked polymer formed by NATURAL POLYMER POLYMER and NATURAL POLYMER POLYMER is expected to the substantially non-toxic (i.e., biocompatible) and biodegradable (or bioresorbable). Additionally, because the crosslinked NATURAL POLYMER POLYMER and NATURAL POLYMER POLYMER microsphere is formed from two polymers, the mechanical properties, such as compressibility, of the crosslinked molecule are expected to be sufficient for use of the particles as abrasive agents.

The plurality of microspheres described herein may be used for any suitable purpose, e.g., for radioactive embolization. Because the plurality of microspheres are biocompatible and biodegradable, the microspheres may be acceptable for use within the body, and may degrade after use, which may reduce environmental contamination by the microspheres.

The ingredient may include, for example, a therapeutic or diagnostic radionuclide, optionally in combination with one or more additional ingredients, such as an antibiotic, antimicrobial, antifungal, or the like. For example, the ingredient may include a therapeutic radionuclide, such as yttrium-90.

In some examples, the microspheres comprising NATURAL POLYMER POLYMER and NATURAL POLYMER POLYMER may be formed according to the technique described in U.S. Pat. No. 8,617,132, the disclosure of which is incorporated herein by reference. Initially, NATURAL POLYMER POLYMER is at least partially oxidized to form partially oxidized NATURAL POLYMER POLYMER. In one reaction a single NATURAL POLYMER POLYMER monomer (repeating unit), which is part of a chain comprising n repeating units, is reacted with NaIO4 (sodium periodate to oxidize the C-C bond between carbon atoms bonded to hydroxyl groups to form carbonyl (more particularly aldehyde) groups. In some examples, the reaction may be carried out at about 250C. Some or all repeating units within the NATURAL POLYMER POLYMER polymer may be oxidized. For example, some repeating units may not be oxidized at all, and may still include two hydroxyl groups the reaction is performed. Other monomers may be oxidized, and may include two carbonyl groups. The NATURAL POLYMER POLYMER may include a weight average molecular weight of between about 50,000 daltons (Da; equivalent to grams per mole (g/mol)) and about 800,000 Da. In some examples, a weight average molecular weight of the NATURAL POLYMER POLYMER may be about 700,000 g/mol.

The degree of oxidation of the NATURAL POLYMER POLYMER may be affected by, for example, the molar ratio of NaIO4 to NATURAL POLYMER POLYMER repeating units. In some examples, the molar ratio of NaIO4 molecules to NATURAL POLYMER POLYMER repeating units may be between about 0.1:1 and about 0.5:1 (NaIO4:NATURAL POLYMER POLYMER repeating unit).

Particular examples of molar ratios of NalO4 molecules to NATURAL POLYMER POLYMER repeating units include about 0.1:1, about 0.25:1, and about 0.5:1. An increased molar ratio of NalO4 molecules to NATURAL POLYMER POLYMER repeating units may result in greater oxidation of the NATURAL POLYMER POLYMER, which in turn may lead to greater crosslinking density when NATURAL POLYMER POLYMER is reacted with NATURAL POLYMER POLYMER to form the microspheres. Conversely, a decreased molar ratio of NalO4 molecules to NATURAL POLYMER POLYMER repeating units may result in lesser oxidation of the NATURAL POLYMER POLYMER, which in turn may lead to lower crosslinking density when NATURAL POLYMER POLYMER is reacted with NATURAL POLYMER POLYMER to form the microspheres. In some examples, the crosslinking density may be approximately proportional to the degree of oxidation of the NATURAL POLYMER POLYMER. In some examples, a greater crosslinking density may lead to microspheres with greater mechanical strength (e.g., fracture strain).

NATURAL POLYMER POLYMER may be prepared by reacting chitosan to attach —CH2COO— groups in place of one of the hydrogen atoms in an amine group or a hydroxyl group, as illustrated in Reaction 2 of the above-cited '132 patent. In the product of Reaction 2, each R is independently either H or —CH2COO—. Similar to oxidation of NATURAL POLYMER POLYMER shown in Reaction 1, the extent of the addition of the —CH2COO— may affect the crosslink density when the NATURAL POLYMER POLYMER is reacted with the partially oxidized NATURAL POLYMER POLYMER to form the microspheres. The extent of the addition of the —CH2COO— may be affected, for example, by the ratio of ClCH2COOH to NATURAL POLYMER POLYMER repeating units. In general, a greater ratio of —CH2COO— to NATURAL POLYMER POLYMER repeating units may result in a greater extent of the addition of —CH2COO—, which a lesser ratio of —CH2COO— to NATURAL POLYMER POLYMER repeating units may result in a lesser extent of the addition of —CH2COO—.

In some examples, the ratio of x:y in the NATURAL POLYMER POLYMER may be about 3:1 (i.e., monomers of “x” form about 75% of the chitosan and monomers of “y” form about 25% of the chitosan), although other ratios may also be used. In some examples, the chitosan starting material may have a molecular weight between about 190,000 g/mol and about 375,000 g/mol. In some examples, Reaction 2 may be performed by stirring the reaction mixture at 500 rpm for about 24 hours at about 25° C., followed by stirring the reaction mixture at 500 rpm for about 4 hours at about 50° C.

Once the partially oxidized NATURAL POLYMER POLYMER and the NATURAL POLYMER POLYMER have been prepared, each is mixed in a respective amount of a solvent, such as water. For example, 0.1 milligram (mg) of partially oxidized NATURAL POLYMER POLYMER may be mixed in 5 milliliter (mL) of water to form a first 2% weight/volume (w/v) solution. Similarly, 0.1 mg of NATURAL POLYMER POLYMER may be mixed in 5 mL of water to form a second 2% w/v solution. Of course, solvents other than water may be used, and solutions having other concentrations of partially oxidized NATURAL POLYMER POLYMER or NATURAL POLYMER POLYMER, respectively, may be utilized. For example, saline or phosphate-buffered saline (PBS) may be utilized as alternative solvents. The solvent used in the partially oxidized NATURAL POLYMER POLYMER solution may be the same as or different than the solvent used in the NATURAL POLYMER POLYMER solution. The solutions may have concentrations of partially oxidized NATURAL POLYMER POLYMER or NATURAL POLYMER POLYMER between about 0.5% w/v and about 3% w/v. The concentration of the partially oxidized NATURAL POLYMER POLYMER solution may be the same as or different from the concentration of the NATURAL POLYMER POLYMER solution.

As discussed above, the crosslinking reaction of the NATURAL POLYMER POLYMER and NATURAL POLYMER POLYMER may proceed without use of a small-molecule crosslinking agent, such as glutaraldehyde. This may be advantageous, because in some examples, a small-molecule crosslinking agent may be toxic to a patient which uses products including the microspheres. In this way, the microspheres formed from NATURAL POLYMER POLYMER crosslinked with NATURAL POLYMER POLYMER may be substantially free of any small-molecule crosslinking agent.

In some examples, the crosslinking reaction between NATURAL POLYMER POLYMER and NATURAL POLYMER POLYMER may proceed under relatively benign conditions. For example, the crosslinking reaction may be carried out at ambient pressures and ambient temperatures (e.g., room temperature). In some examples, the reaction may be carried out at a temperature above ambient, such as, for example, 40° C. Example ranges of temperatures in which the crosslinking reaction may be performed include between about 20° C. and about 70° C., and at about 40° C. or about 65° C. In some examples, a lower reaction temperature may necessitate a longer reaction time to result in substantially similar diameter microspheres, or may result in smaller microspheres after a similar amount of time.

One advantage of performing the reaction at a temperature above room temperature may be the removal of water from the reaction mixture during the course of the reaction. For example, performing the crosslinking reaction at a temperature of about 65° C. may result in evaporation of water as the crosslinking reaction proceeds.

An extent of crosslinking between molecules of NATURAL POLYMER POLYMER and NATURAL POLYMER POLYMER may affect mechanical properties of the resulting microsphere. For example, a greater crosslinking density generally may provide greater mechanical strength (e.g., fracture strain), while a lower crosslinking density may provide lower mechanical strength (e.g., fracture strain). In some examples, the crosslinking density may be adjustable to provide a fracture strain of between about 70% and about 90%, as described below with respect to FIG. 7. The crosslinking density may also affect the degradation rate of the microsphere. For example, a greater crosslinking density may lead to a longer degradation time, while a lower crosslinking density may lead to a shorter degradation time. In some examples, the crosslink bonds may degrade through hydrolyzing of the C═N double bond.

As described above, the crosslinking reaction between NATURAL POLYMER POLYMER and NATURAL POLYMER POLYMER is a modified emulsion-crosslinking reaction. In some examples, an emulsion-crosslinking reaction may be rate-limited by transport of the NATURAL POLYMER POLYMER and NATURAL POLYMER POLYMER molecules, and may play a role in the reaction product (the crosslinked NATURAL POLYMER POLYMER and NATURAL POLYMER POLYMER) being microspheres.

The size of the microspheres may be affected by reaction conditions, such as, for example, a stirring speed, a reaction temperature, a concentration of the NATURAL POLYMER POLYMER and NATURAL POLYMER POLYMER molecules in the reaction emulsion, an amount of mixing of the emulsion, or a concentration of the surfactant in the emulsion. For example, increasing the concentration of each of the NATURAL POLYMER POLYMER and NATURAL POLYMER POLYMER solutions from 1.5% w/v to 2% w/v while keeping the oxidation degree of NATURAL POLYMER POLYMER at about 25% (about 25 oxidized repeating units per 100 total repeating units), the stirring speed at 600 revolutions per minute (rpm), the temperature at about 50C, the reaction time at about 12 hours, and the amount of Span 80 at about 0.3 mL/50 mL mineral oil, the average diameter of the microspheres may increase from about 600 μm to about 1100 μm. As another example, increasing the oxidation degree of NATURAL POLYMER POLYMER from about 10% to about 25% while keeping the concentration of each of the NATURAL POLYMER POLYMER and NATURAL POLYMER POLYMER solutions at about 1.5% w/v, the stirring speed at 600 rpm, the temperature at about 50C, the reaction time at about 12 hours, and the amount of Span 80 at about 0.3 mL/50 mL mineral oil, the average diameter of the microspheres may increase from about 510 μm to about 600 μm.

In some examples, the reaction conditions may be selected to result in microspheres with a mean or median diameter between about 40 μm and about 2200 μm. In some examples, the reaction conditions may be selected to result in microspheres with a mean or median diameter of less than about 2000 μm, microspheres with a mean or median diameter of between about 100 μm and about 1200 μm, microspheres with a mean or median diameter of between about 100 μm and about 300 μm, microspheres with a mean or median diameter of between about 300μm and about 500 μm, microspheres with a mean or median diameter of between about 500 μm and about 700 μm, microspheres with a mean or median diameter of between about 700 μm and about 900 μm, microspheres with a mean or median diameter of between about 900 μm and about 1200 μm, or microspheres with a mean or median diameter of between about 1600 μm and about 2200 μm. In some examples, the diameter of the microspheres may be measured using optical microscopy, approximated based on using one or more sieves, or the like.

Once the reaction has proceeded for a desired amount of time to produce microspheres with a desired mean or median diameter, the water in the emulsion may be substantially fully removed, if the water has not already been evaporated during the crosslinking reaction. The oil phase may then be removed, such as by decanting or centrifugation, and the microspheres may be washed. For example, the microspheres may be washed with Tween 80 solution. Finally, the microspheres may be stored in a liquid, such as water or saline, at a suitable temperature, such as between about 2° C. and about 8° C.

In some examples, the crosslinking reaction may produce a plurality of microspheres with diameters distributed about a mean or median. In some cases, it may be advantageous to isolate microspheres with diameters within a smaller range or microspheres with substantially a single diameter. In some examples, the microspheres may be separated according to diameter by wet sieving in normal saline through a sieve or sieves with predetermined mesh size(s).

This invention relates to a crosslinked NATURAL POLYMER POLYMER/NATURAL POLYMER POLYMER microsphere that comprises a polymer, particularly a polymer and a radionuclide, as well as to a method for the production thereof, and to methods for the use of this particulate material. In one particular aspect, this invention relates to microspheres which comprise a polymer and a radionuclide such as radioactive yttrium, and to the use of these microspheres in the treatment of cancer and related conditions in humans and other mammals. See, for instance, WO2002034300, the disclosure of which is incorporated herein by reference.

The crosslinked NATURAL POLYMER POLYMER/NATURAL POLYMER POLYMER microsphere of this invention can be designed to be administered into the arterial blood supply of an organ to be treated, whereby it becomes entrapped in the small blood vessels of the target organ and irradiates it. An alternate form of administration is to inject the polymer based crosslinked NATURAL POLYMER POLYMER/NATURAL POLYMER POLYMER microsphere directly into the target organ or a solid tumor to be treated.

The crosslinked NATURAL POLYMER POLYMER/NATURAL POLYMER POLYMER microsphere of the present invention therefore has utility in the treatment of various forms of cancer and tumors, but particularly in the treatment of primary and secondary cancer of the liver and the brain. When microspheres or other small particles are administered into the arterial blood supply of a target organ, it is desirable to have them of a size, shape and density that results in the optimal homogeneous distribution within the target organ. If the microspheres or small particles do not distribute evenly, and as a function of the absolute arterial blood flow, then they may accumulate in excessive numbers in some areas and cause focal areas of excessive radiation. It has been shown that microspheres of about 25 microns to about 50 microns in diameter have the best distribution characteristics when administered into the arterial circulation of the liver.

If the particles are too dense or heavy, then they will not distribute evenly in the target organ and will accumulate in excessive concentrations in areas that do not contain the cancer. It has been shown that solid, heavy microspheres distribute poorly within the parenchyma of the liver when injected into the arterial supply of the liver. This, in turn, decreases the effective radiation reaching the cancer in the target organ, which decreases the ability of the radioactive microspheres to kill the tumor cells.

For radioactive crosslinked NATURAL POLYMER POLYMER/NATURAL POLYMER POLYMER microsphere to be used successfully for the treatment of cancer, the radiation emitted should be of high energy and short range. This ensures that the energy emitted will be deposited into the tissues immediately around the crosslinked NATURAL POLYMER POLYMER/NATURAL POLYMER POLYMER microsphere and not into tissues which are not the target of the radiation treatment. In this treatment mode, it is desirable to have high energy but short penetration beta-radiation which will confine the radiation effects to the immediate vicinity of the particulate material. There are many radionuclides that can be incorporated into microspheres that can be used for SIRT. Of particular suitability for use in this form of treatment is the unstable isotope of yttrium (Y-90).

Yttrium-90 decays with a half life of 64 hours, while emitting a high energy pure beta radiation. However, other radionuclides may also be used in place of yttrium-90 of which the isotopes of holmium, samarium, iodine, iridium, phosphorus, rhenium are some examples.

Microspheres of this invention can be provided using any suitable means. See, for instance, Serra et al., 2013 (cited above), the disclosure of which is incorporated herein by reference. For instance, they can be prepared by either heterogeneous polymerization processes (suspension, supercritical fluid) or by precipitation processes in a non-solvent. Preferably, however, the microspheres are prepared using microfabrication techniques that enable the preparation of very efficient emulsification microstructured devices which, along with capillaries of small dimensions, allow emulsifying a fluid in another immiscible fluid. Thus, droplets or bubbles, with an extremely narrow size distribution (the coefficient of variation of the particle size distribution is typically lower than 5%) can be continuously produced and dispersed in a continuous fluid flowing within these microfluidic devices. If the ‘to be dispersed’ phase is composed of a polymerizable liquid, the droplets can be hardened downstream either by thermally or photo-induced polymerization. Over conventional processes, microfluidic-assisted processes offer the possibility not only to precisely control the size of the particle but also its shape, morphology and composition. At least two different categories of microsystem are suitable for the emulsification of a polymerizable liquid. In the first one, both continuous and dispersed fluids flow inside microchannels, while in the second one, the continuous phase flows inside a tube and the dispersed phase inside a capillary of small dimensions. The emulsification mechanism, which is quite similar for these two categories of microsystem, proceeds from the break-up of a liquid thread into droplets when the to-be-dispersed phase is sheared by the continuous and immiscible phase.

A variety of microchannel-based devices can be used, including for instance, a terrace-like microchannel device, a T-junction microchannel device, and a flow-focusing microchannel device. These devices are usually microfabricated, thanks to semiconductor related like technologies. Thus, lithographic processes are commonly employed to etch into silicon, glass, or polydimethylsiloxane (PDMS) microchannels in which the continuous and dispersed phases flow. Over capillary-based devices, microchannel-based systems offer some unique features. Microsystems with channel widths as low as few tens of microns can be obtained. Mask lithographic techniques allow for a perfect alignment of the microchannels and complex microstructures.

Upstream and downstream functionalities (flow distribution, selective droplets fusion, droplet scissions, etc.) are easily implemented. Finally, chips with multiple microstructures can be designed for increasing the overall production of polymer particles.

A variety of capillary-based devices can also be used, including a co-flow capillary device, a cross-flow capillary device, and a flow-focusing capillary device. All the above microchannel-based devices are designed such that the dispersed phase is in direct contact with the wall of the device before being emulsified by the continuous phase. So the device material should be carefully chosen or modified to avoid a phase inversion. This phenomenon is observed when the dispersed phase has a greater affinity for the material than the continuous phase; i.e., when the dispersed phase wets preferentially the walls. As a result, the continuous phase is emulsified by the dispersed phase and droplets of continuous phase are formed. This phase inversion can be avoided by selecting a proper material hydrophilic for hydrophobic droplets) or by modifying locally the properties of the material at the very location where droplets of dispersed phase are formed. However, the latter procedure requires an additional step in the microfabrication process. Additionally, one can use capillary-based devices to deliver the dispersed phase in the very center line of the continuous phase flow so that the droplets never meet with the device walls. Moreover, these capillary based devices solve for the clogging of microchannels that can be encountered in the above microchannel-based devices as well as for the possibility to get O/W or W/O emulsion with a single microsystem.

Simple morphologies like beads and capsules can be obtained from the abovementioned microfluidic devices. However, in addition to the greater control over the size, these devices also allow for the production of specific polymer particles, which characteristics (morphology and composition) are likely to be difficult to obtain in conventional batch reactors dispersed phase has a greater affinity for the material than the continuous phase, i.e., when the dispersed phase wets preferentially the walls. As a result, the continuous phase is emulsified by the dispersed phase and droplets of continuous phase are formed. This phase inversion can be avoided by selecting a proper material size and size distribution were obtained by analyzing up to 50 particle optical micrographs by means of an imaging software.

Droplet size can be controlled by various means, including in articular operating parameters such as dispersed and continuous velocities, internal capillary diameter, the viscosity of dispersed and continuous phases, and surface tension. In one example, and preferred embodiment, the microspheres are provided by the application of a capillary-based microsystem that allows the preparation of polymeric microparticles of different shapes (e.g., spheres and rods) and/or with different morphologies (e.g., Janus and core-shell particles).

Capillary-based microsystems were found very convenient to produce polymeric capsules (average size of 300 μm) and to investigate effect of operating and composition parameters on the morphology of the membrane. These parameters can be easily changed, and a small amount as low as 1 mL of the dispersed phase is required to investigate capsules characteristics.

Given the present description, those skilled in the art will be able to prepare polymeric materials according to the present invention in any suitable form, e.g., in the form of spherical or janus-like microparticles. These microparticles exhibit some specific properties which arise from either the narrow size distribution or from their morphology that cannot be achieved when they are prepared by more conventional synthetic methods.

### EXAMPLES

### Example 1

### Preparation of Yttrium-Containing Microspheres by Emulsion

Partially Oxidize Natural Polymer polymers are prepared in the manner described in Examples 1 and 4 of U.S. Pat. No. 8,617,132, the disclosure of which is incorporated herein by reference. About 0.075 g of polymer 1 is mixed in about 5 mL of water to form a 1.5% w/v polymer 2 solution. Similarly, about 0.075 g ONATURAL POLYMER POLYMER-II is mixed in about 5 ml water to form a 1.5% w/v ONATURAL POLYMER POLYMER-II solution. The two polymeric solutions are then mixed. Yttrium-90 is obtained by irradiating Yttrium oxide to produce yttrium-90 from the nuclear reaction Y-89 (n, y) Y-90. Yttrium-90 has a half life of 64 hours. The yttrium (90Y) oxide is then dissolved in 0.1 M sulphuric acid with gentle heating and stirring to form a clear, colourless solution of yttrium (90Y) sulphate. The Yttrium (90Y) sulphate is incorporated into the polymer solution and the mixture is used as the dispersed phase. The amount of yttrium (90Y) sulphate that added is determined by limiting the radioactively of each microsphere in the range of 3.75-7.5x10-8 GBq. The mixture is added to about 50 mL mineral oil containing between 0.2 mL and 0.5 mL sorbitane monooleate to form an emulsion, and the emulsion is homogenized for about 15 minutes. The mixture is then stirred overnight at 40-60C to form crosslinked microspheres. Then oil is decanted, and the microspheres can be washed with 5% Tween 80 followed by 0.9% saline.

The mean diameter of the microspheres, measured in normal saline by a light microscope, is determined to be between 20 and 60 microns in diameter. The maximum energy of the beta particles is 2.27 MeV, and the maximum range of emissions in tissue is between about 2 and 15 mm. The half life is 64.1 hours. In therapeutic use, requiring the isotope to decay to infinity, 94% of the radiation is delivered within about 7 to about 11 days. The polymer matrix is substantially bioresorbed within 15 to 20 days.

### Example 2

### Preparation of Yttrium-Containing Microspheres by Droplet Microfluidics

Partially Oxidized NATURAL POLYMER POLYMER and O are prepared in the manner described in Examples 2 and 4 of U.S. Pat. No. 8,617,132, the disclosure of which is incorporated herein by reference. About 0.075 g of NATURAL POLYMER POLYMER-I is mixed in about 5 mL of water to form a 1.5% w/v NATURAL POLYMER POLYMER-I solution. Similarly, about 0.075 g ONATURAL POLYMER POLYMER-I is mixed in about 5 ml water to form a 1.5% w/v ONATURAL POLYMER POLYMER-I solution. The NATURAL POLYMER POLYMER-I and NATURAL POLYMER POLYMER-I solutions are then mixed. Yttrium-90 is obtained by irradiating Yttrium oxide to produce yttrium-90 from the nuclear reaction Y-89 (n, y) Y-90. The yttrium (90Y) oxide is then dissolved in 0.1 M sulphuric acid with gentle heating and stirring to form a clear, colourless solution of yttrium (90Y) sulphate. The Yttrium (90Y) sulphate is incorporated into the polymer solution and the mixture will be used as the disperse phase. The amount of Yttrium (90Y) sulphate added is determined by limiting the radioactively of each microsphere in the range of 3.75-7.5×10-8 GBq. Mineral oil containing between 0.4-1% sorbitane monooleate will be used as a continuous phase. Microspheres in the size range of 20-60 μm are prepared with the co-flow capillary-based microsystem (Serra et al., 2013).

Microdroplets and subsequent yttrium core polymer shell microparticles are obtained from capillary based microfluidic devices consisting in different arrangement of capillaries single, co-axial, and side-by-side having small inner diameters (ca. 20-150 μm). Either of two devices can be used, including a co-flow and a flow-focusing microsystem. At the capillary tip, the to-be-dispersed phase, composed of a monomer solution admixed with an initiator, is sheared by the continuous phase to form, in the dripping regime, droplets of same volume with a regular frequency up to several tens of Hz. Depending on the capillaries arrangement, single, double, or janus droplets are produced. All microsystems are composed of capillaries with hydrophilic or hydrophobic inner walls, T-junctions and tubing.

Formation of droplet is observed under an optical microscope equipped with a CCD camera capturing up to 200 fps at a full resolution of 648×488 pixels. Application of these capillary-based microsystems allows the preparation of polymeric microparticles of different shapes including spheres and rods. One can also produce microparticles with different morphologies, including janus and core-shell whose shell thickness can be tuned simply by adjusting the operating conditions (mainly continuous and dispersed phase flow rates).

The preformed microspheres are collected in a container with the mineral oil and the aqueous phase of the emulsion is allowed to evaporate over night at about 40-60C with constant stirring. Then microspheres will be filtered, and washed with 5% Tween 80 followed by 0.9% saline.

1. A composition comprising crosslinked NATURAL POLYMER POLYMER/NATURAL POLYMER POLYMER microspheres comprising a stably incorporated radionuclide.

2. The composition according to claim 1, wherein the radionuclide comprises yttrium-90.

3. The composition according to claim 1, wherein the microsphere has been prepared by droplet microfluidics.

4. The composition according to claim 1, wherein the microsphere is adapted to substantially release the radionuclide over a period of between about 7 to about 11 days.

5. The composition according to claim 1, wherein the polymer matrix is substantially bioresorbed within 15 to 20 days.

6. A method of making a composition comprising crosslinked NATURAL POLYMER POLYMER/NATURAL POLYMER POLYMER microspheres, the method selected from a group consisting of emulsion formation and droplet microfluidics.

7. A method of treating of a patient's body, comprising the steps of providing a composition comprising crosslinked NATURAL POLYMER POLYMER/NATURAL POLYMER POLYMER microspheres, and delivering the composition to a site within the body.
